(19) United States
(12) Patent Application Publication
(10) Pub. No.: US 2015/0320998 A1
(43) Pub. Date: Nov. 12, 2015

(54) Device, system and method for killing viruses in blood

(71) Applicant: Dan Hester, Chattanooga, TN (US)

(72) Inventor: Dan Hester, Chattanooga, TN (US)

(21) Appl. No.: 14/704,970

(22) Filed: May 6, 2015

Related U.S. Application Data
(60) Provisional application No. 61/989,880, filed on May 7, 2014.

(51) Int. Cl. A61N 1/30; A61B 8/00; A61M 25/01

(52) U.S. Cl. CPC A61N 1/306; A61M 25/01; A61B 8/565; A61M 2205/3592; A61M 2205/52

(57) ABSTRACT

A device, system and method for killing viruses in blood. An iontophoretic terminal delivery system includes a smart catheter, aleated electrode within the smart catheter, a terminal and an electrical system. Ultrasound cannulation is used to guide the smart catheter of the iontophoretic terminal delivery system into a vein of the patient. Electrical power is provided to the aleated electrode of the smart catheter, thereby releasing positively charged ionized silver nanoparticles into the blood stream that attract negatively charged viruses in order to effectively destroy them. The smart catheter includes two micro fluid chips, a Lab On Chip that counts viral loads as well as patient progress in real time, and a Polymerase Chain Reaction chip, which checks for other viruses or infections. Collected data is passed to the terminal where it is stored and made available in a digitized format. Together these chips form a redundant biosensing system.

### CROSS REFERENCE TO RELATED APPLICATIONS

This application claims the benefit of U.S. Provisional Pat. App. No. 61/989,880 filed on May 7, 2014, the entirety of which is hereby incorporated by reference.

### BACKGROUND OF THE INVENTION

### Field of the Invention

This invention relates to the general field of medical devices and methods, and more specifically toward a device, system and method for killing viruses in blood. An iontophoretic terminal delivery system is disclosed herein that includes a smart catheter, aleated electrode within the smart catheter, a terminal and an electrical system. Ultrasound cannulation is used to guide the smart catheter of the iontophoretic terminal delivery system into the subclavian vein of the patient. Electrical power is provided to the aleated electrode of the smart catheter, thereby releasing positively charged ionized silver nanoparticles into the blood stream that attract negatively charged viruses in order to effectively destroy them. The smart catheter includes two micro fluid chips. The first chip is the Lab On Chip (LOC) that counts viral loads as well as patient progress in real time. The second chip is a Polymerase Chain Reaction (PCR) chip, which checks for other viruses or infections. As the data is collected it is passed to the terminal where it is stored and made available to the health care provider in a digitized format. Together these chips form a redundant biosensing system.

Viral infections of the blood can have a devastating impact on a patient once the virus has infected its host. In many cases it ends with the death of the host. The more deadly forms of the blood viruses are HIV/AIDS and Hepatitis C. HIV can mutate quickly and therefore can be very difficult to create an effective vaccine or drug cocktail that works against it. With Hepatitis C, there are many different strains of the virus and therefore drug therapies that are available can be non-effective and/or have debilitating side effects. The patients that the drugs do not work on are classified as non-responders and there chances for survival are very bleak. One particular strain of Hepatitis C has been linked to a new form of liver cancer that has not been seen before. There has also been a rise in Hepatitis C worldwide due to the popularity of tattooing and the lack of proper hygiene associated with it. The only proven way to destroy these blood viruses is with the use of silver ions.

Silver iontophoresis is a physical process wherein silver ions are driven by an electrical field and flow diffusively through a medium. The prior art has used silver iontophoresis by inserting a catheter into the subclavian vein or the superior vena cava and then placing a silver probe through it and directly into the blood stream. A small electrical current is then applied to the wire in the prescribed amount to release the proper amount of silver nanoparticles, which have a slightly positive charge, to bond to viruses, which has a slightly negative charge. This process destroys the virus by disrupting the functions of the membrane of the virus and thus its ability to survive. This procedure has various challenges associated with it due to its close proximity to the heart, its duration of time needed to be successful and its chances of creating a secondary infection at the entry site.

Thus there has existed a long-felt need for a device and method that efficiently and safely kills viruses in blood.

### SUMMARY OF THE INVENTION

The current invention provides just such a solution by having an iontophoretic terminal delivery system is disclosed herein that includes a smart catheter, aleated electrode within the smart catheter, a terminal and an electrical system. Ultrasound cannulation is used to guide the smart catheter of the iontophoretic terminal delivery system into the subclavian vein of the patient. Power is provided to the aleated electrode of the smart catheter, thereby releasing positively charged ionized silver nanoparticles into the blood stream that attract negatively charged viruses in order to effectively destroy them. The smart catheter includes two micro fluid chips. The first chip is the LOC that counts viral loads as well as patient progress in real time. As the data is collected it is passed to the terminal where it is stored and made available to the health care provider in a digitized format. The second chip is a PCR chip, which checks for other viruses or infections. Together these chips form a redundant biosensing system.

Cannulation of veins and arteries is an important aspect of patient care for the administration of fluids and medications, as well as for monitoring purposes. The practice of using surface anatomy and palpation to identify target vessels before cannulation attempts is based on the presumed location of the vessel, the identification of surface or skin anatomic landmarks, and blind insertion of the needle until blood is aspirated.

In a particular embodiment, the method disclosed herein incorporates the use of ultrasound cannulation to safely guide the smart catheter in to the subclavian vein where the Iontophoretic process takes place. The most common complication of subclavian vein cannulation is pneumothorax. The incidence of mechanical complications increases six fold when more than three attempts are made by the same operator. The use of ultrasound imaging before and/or during vascular cannulation greatly improves first-pass success and reduces complications. Practice recommendations for the use of ultrasound for vascular cannulation have emerged from numerous specialties, governmental agencies such as the National Institute for Health and Clinical Excellence and the Agency for Healthcare Research and Quality's evidence report.

The iontophoretic terminal delivery system enables care givers to treat and monitor patients during iontophoretic procedures in real time. Not only does the iontophoretic terminal delivery system monitor iontophoresis, it also analyzes the blood of the patient infections and other diseases. Furthermore, once inserted into the subclavian vein, the iontophoretic terminal delivery system is portable and gives the patient greater mobility and reduces convalescence time. Since patients' physiologies are different, real time biomonitoring gives health care providers more flexibility in tailoring treatment for a specific patient.

It is an object of the invention to provide a device for killing viruses in blood.

It is another object of the invention to provide a method for killing viruses in blood.

It is a further object of this invention to provide a device and method for treating blood without removing it from the body.

As used herein, the term “patient” refers not only to a human, but also to mammals and even animals in general; the terms “rod” or “wire” refer to a thin, straight, and rigid or flexible bar; the term “aleated” means coated or insulated except for a small portion.

A particular embodiment of the current disclosure has a system for treating a viral infection within a blood stream comprising a smart catheter and a terminal; where the smart catheter comprises an aleated electrode, lab on chip and a polymerase chain reaction chip, where the aleated electrode comprises a silver nanoparticle tip; where the terminal comprises a central processing unit, electronic memory, and a data port, where the terminal is electrically connected to the smart catheter, where the terminal provides electrical power to the aleated electrode of the smart catheter, and where data collected by the lab on chip, the polymerase chain reaction chip, or both is transmitted to the terminal.

Another embodiment of the current disclosure provides a method for treating a viral infection within a blood stream comprising the steps of: inserting a smart catheter into the subclavian vein of a patient, where the smart catheter comprises an aleated electrode, lab on chip and a polymerase chain reaction chip, where the aleated electrode comprises a silver nanoparticle tip; providing power to the aleated electrode of the smart catheter; and transmitting data from the lab on chip, the polymerase chain reaction chip, or both to a terminal, where the terminal comprises a central processing unit, electronic memory, and a data port.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims.

### BRIEF DESCRIPTION OF THE FIGURES

The accompanying drawings, which are incorporated in and form a part of this specification, illustrate embodiments of the invention and together with the description, serve to explain the principles of this invention.

FIG. 1 is a schematic view of a smart catheter of an iontophoretic terminal delivery system according to selected embodiments of the current disclosure.

FIG. 2 is a process chart of the LOC and PCR chip sensors according to selected embodiments of the current disclosure.

FIG. 3 is a diagram of a motherboard of the terminal of the iontophoretic terminal delivery system according to selected embodiments of the current disclosure.

FIG. 4 is a front view of a patient showing the location of insertion of the smart catheter into the subclavian vein according to selected embodiments of the current disclosure.

FIG. 5 is diagram showing the insertion of the smart catheter into the subclavian vein according to selected embodiments of the current disclosure.

### DETAILED DESCRIPTION OF THE INVENTION

Many aspects of the invention can be better understood with the references made to the drawings below. The components in the drawings are not necessarily drawn to scale. Instead, emphasis is placed upon clearly illustrating the components of the present invention. Moreover, like reference numerals designate corresponding parts through the several views in the drawings.

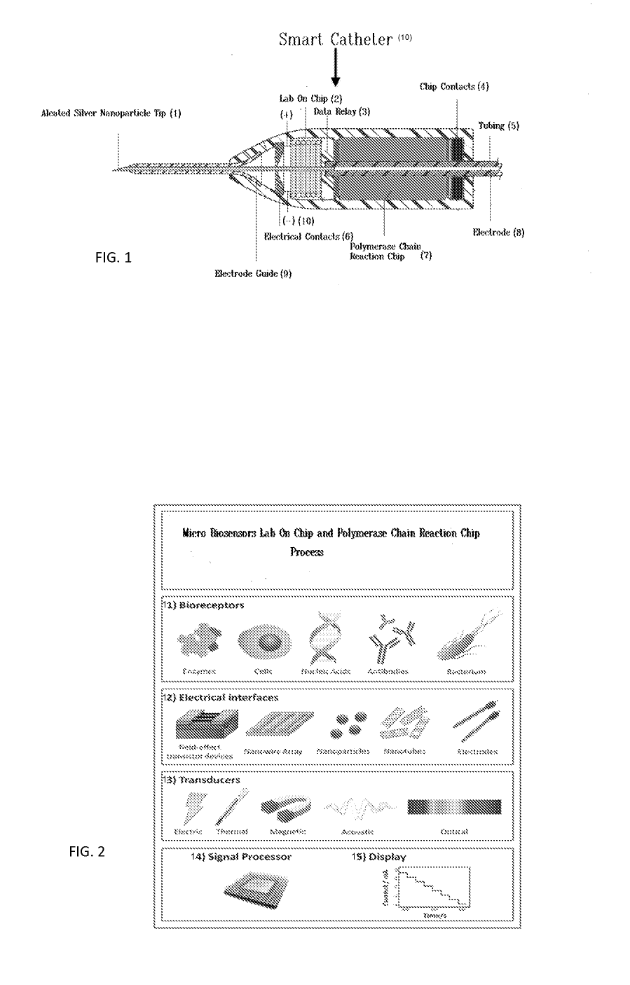

FIG. 1 is a schematic view of a smart catheter of an iontophoretic terminal delivery system according to selected embodiments of the current disclosure. The smart catheter (10) includes an electrode (8) within tubing (5). The electrode has an aleated silver nanoparticle tip (1). An electrode guide (6) provides support for the aleated silver nanoparticle tip 1 relative to the rest of the smart catheter (10). A lab on chip (LOC) (2) and polymerase chain reaction chip (PCR) chip (7) are housed within the smart catheter (10), and discussed in more detail below. Data relay (3) and chip contacts (4) provide electrical and data connections between the LOC and PCR chip as well as to an external terminal (not shown in this figure). Electrical contacts (6) provide an electrical connection to the electrode (8) and aleated silver nanoparticle tip (1).

The electrode (8) is covered in polytetrafluoroethylene (PTFE) (Teflon®), except for the bare tip, which is where the silver nanoparticles are located and await a 5 μA charge thereby releasing the positively charged ionized silver nanoparticles that attract the negatively charged viruses in order to effectively destroy them.

FIG. 2 is a process chart of the LOC and PCR chip sensors according to selected embodiments of the current disclosure. The LOC and PCR chip biosensors have bioreceptors (11) to detect enzymes, cells, nucleic acids, antibodies and bacterium. These bioreceptors interact with (12) electrical interfaces and (13) transducers to provide electrical signals to a signal processor (14). The signal processor (14) then provides modified electrical signals to an external device, such as a display (15), terminal, or other device.

In a particular embodiment, a typical biosensor is composed of five parts as illustrated in bio-receptors (11) that bind of specific form to the analyte; electrochemically active interfaces (12) where specific biological process occur giving rise to a signal; a transducer element (13) that converts the specific biochemical reaction in an electrical signal that is amplified by a detector circuit using the appropriate reference; a signal processor (14) (e.g. computer software) for converting the electronic signal to a meaningful physical parameter describing the process being investigated and finally, a proper interface (15) to present the results to the human operator.

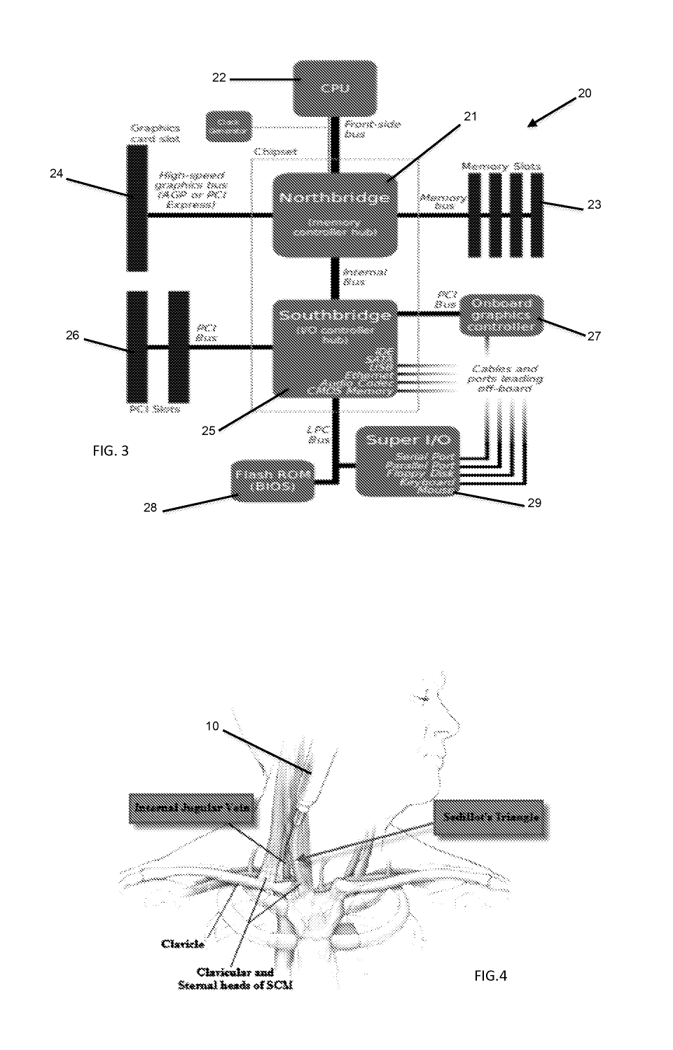

FIG. 3 is a diagram of a motherboard of the terminal of the iontophoretic terminal delivery system according to selected embodiments of the current disclosure. The motherboard (20) includes a northbridge (21), which interacts with the central processing unit (CPU), (22), memory bus (23), and the graphics bus (24). The northbridge (21) is also connected to the southbridge (25), which interacts with the peripheral component interconnect (PCI) bus, graphics controller 27, flash read only member (ROM), and other input/output ports (29).

The terminal is the backbone of the iontophoretic terminal delivery system. All data gathered from the LOC and PCR chip will be stored and accessible from various end user devices, such as a phone, laptop or desktop. In essence the terminal is the black box for the iontophoretic terminal delivery system.

A motherboard is the most essential part of the terminal. The motherboard enables the terminal to attach seamlessly to a local area network (LAN) or a wireless local area network (WLAN). Input/ouput ports and interfaces of the terminal include universal serial bus (USB) and Ethernet ports. These data ports enable the device to provide and distribute data in real time. Furthermore, motherboard provides the electrical connections by which the other components of the system communicate (talk with each other), it also contains the central processing unit and hosts other subsystems and devices.

As will be appreciated by those skillied in the art, electrical components are integrated into the motherboard. These parts include transistors and resistors. Important functionality provided by the motherboard me implemented as software or firmware such that it can be upgraded in the future through software/firmware updates. The CPU is the hardware within the terminal that carries out the instructions of a computer program by performing the basic arithmetical, logical, and input/output operations of the system.

In a particular embodiment, the terminal includes multiple USB 2.0 ports, an audio port (such as a 3.5 mm headphone port), a power button, status lights, Serial ATA port, gigabit Ethernet port, high-definition multimedia interface (HDMI) port, video graphics array (VGA) port, a complementary metal-oxide-semiconductor (CMOS) battery, and a heat sink and fan. The heat sink and fan provide cooling to the terminal to resist overheating, which is a considerable factor given the small size of the terminal. Electronic communication applications may be incorporated into the hardware of the terminal to connect the device with a remote healthcare professional. Such electronic communication applications may therefore enable to view data and statistics regarding the patient, as well as change the parameters and settings of the device to provide customized health care from a remote location.

To provide a convenient and non-obtrusive device and system, the terminal should be as small as possible. In a particular embodiment, the terminal has a length of about four inches, a width of about three inches, and a thickness of about three-quarters of an inch.

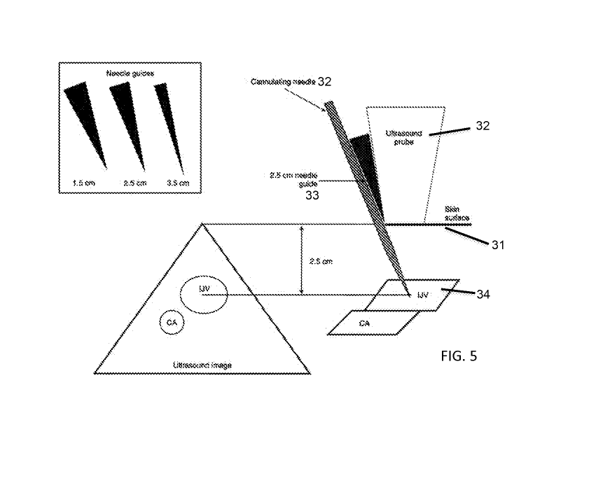

FIG. 4 is a front view of a patient showing the location of insertion of the smart catheter 10 into the subclavian vein according to selected embodiments of the current disclosure.

FIG. 5 is diagram showing the insertion of the smart catheter into the subclavian vein according to selected embodiments of the current disclosure. An ultrasound probe (30) is used to aid a medical practitioner in placing a cannulating needle (32) in the subclavian vein (34). A needle guide (33) placed between the ultrasound probe (30) and cannulating needle (32) also aids in the placement of the smart catheter.

In a particular embodiment, power supply consists of a cigarette box size device that has an on/off switch, a three foot long power supply line that connects to the smart catheter containing the silver/copper wire that is inserted in to the subclavian vein. It also has a three foot long ground wire that attaches to the body. It has a 2 μA to a 5 μA switch, a reset button, an on light, an alarm light and an attachment clip.

In another embodiment, power supply includes a variable current input with a locking mechanism to customize it for each patient according to size, weight and medical condition. The power supply also includes a digital display to monitor the patient in real time with body monitors attached to the patient to read vital signs, etc. Alarms for vitals may also be implemented into the power supply. A 9 volt battery may be used to provide the electrical power within the power supply, though other power source options other than a 9 volt battery are possible.

In a further embodiment, a wireless data connection is used between the smart catheter and the terminal. Instead of connecting to the data port of the terminal, the smart catheter a radio transmitter and receiver that can send data to and receive data from a radio of the terminal. Various transmission protocols may be used between the smart catheter and the terminal, including without limitation those specified in the IEEE 802.11 protocols (e.g., Bluetooth® and WiFi®). Without the need for a wired data line, alternative means of powering the smart catheter may be employed, including without limitation a separate wired power source to directly power the smart catheter or to provide power to an integrated battery system of the smart catheter. In this embodiment, the data port of the terminal can be a wireless network card instead of a physical port, such as a universal serial bus (USB) port.

It should be understood that while the preferred embodiments of the invention are described in some detail herein, the present disclosure is made by way of example only and that variations and changes thereto are possible without departing from the subject matter coming within the scope of the invention.

That which is claimed:

1. A system for treating a viral infection within a blood stream comprising a smart catheter and a terminal;
where the smart catheter comprises an aleated electrode, lab on chip and a polymerase chain reaction chip, where the aleated electrode comprises a silver nanoparticle tip;
where the terminal comprises a central processing unit, electronic memory, and a data port, and where data collected by the lab on chip, the polymerase chain reaction chip, or both is transmitted to the terminal.

2. The system of claim 1, wherein the terminal is electrically connected to the smart catheter.

3. The system of claim 2, wherein the terminal provides electrical power to the smart catheter.

4. The system of claim 1, wherein the smart catheter further comprises a radio for transmitting and receiving data.

5. The system of claim 1, wherein the data port of the terminal comprises a radio for transmitting and receiving data.

6. The system of claim 1, wherein the data collected by the lab on chip, the polymerase chain reaction chip, or both is transmitted wirelessly to the terminal.

7. The system of claim 1, wherein the smart catheter further comprises a battery system.

8. The device of claim 1, further comprising an electrical system, where the electrical system provides electrical power directly to the smart catheter.

9. A method for treating a viral infection within a blood stream comprising the steps of:
inserting a smart catheter into a vein of a patient, where the smart catheter comprises an aleated electrode, lab on chip and a polymerase chain reaction chip, where the aleated electrode comprises a silver nanoparticle tip;
providing power to the aleated electrode of the smart catheter; and
transmitting data from the lab on chip, the polymerase chain reaction chip, or both to a terminal, where the terminal comprises a central processing unit, electronic memory, and a data port.

10. The method of claim 9, where the smart catheter is inserted into a subclavian vein of the patient.

11. The method of claim 9, wherein the terminal is electrically connected to the smart catheter.

12. The method of claim 9, further comprising the step of providing electrical power to the smart catheter via the terminal.

13. The method of claim 9, wherein the smart catheter further comprises a radio for transmitting and receiving data.

14. The method of claim 9, wherein the data port of the terminal comprises a radio for transmitting and receiving data.

15. The method of claim 9, wherein the step of transmitting data from the lab on chip, the polymerase chain reaction chip, or both to a terminal is done wirelessly.

16. The system of claim 9, wherein the smart catheter further comprises a battery system, wherein the power provided to the aleated electrode of the smart catheter is from the battery system of the smart catheter.
